(19) United States
(12) Patent Application Publication
(10) Pub. No.: US 2011/0150798 A1
(43) Pub. Date: Jun. 23, 2011

(54) COMPOSITIONS AND METHODS FOR INCREASING CELLULAR FAR AND BLEACHING SKIN

(76) Inventor: Sarah Bacus, Hinsdale, IL (US)

(21) Appl. No.: 12/975,265

(22) Filed: Dec. 21, 2010

Related U.S. Application Data
(60) Provisional application No. 61/288,695, filed on Dec. 21, 2009.
(60) Provisional application No. 61/288,677, filed on Dec. 21, 2009.

(51) Int. Cl. A61K 8/64; A61K 8/49; A61Q 19/02

(52) U.S. Cl. 424/62; 514/369; 514/9.2

(57) ABSTRACT

Compositions and methods are disclosed for the treatment of cells, such as keratinocytes and adipocytes, to increase their lipid content and compositions and methods are disclosed for treatment of skin cells to diminish the appearance of blemishes.

### CROSS REFERENCE TO RELATED APPLICATIONS

This application claims priority to U.S. Provisional Patent Application Nos. 61/288,695 and 61/288,677 both filed on Dec. 21, 2009, entitled “Compositions and Methods for Bleaching Skin” and “compositions and Methods for Increasing Cellular Fat,” respectively, by Sarah Bacus, hereby incorporated by reference.

### TECHNICAL FIELD

The present invention relates to a novel anti-wrinkle composition. More specifically, the invention relates to an anti-wrinkle composition whose active ingredient is a compound that acts as an peroxisome proliferator-activated receptor agonist. The invention further relates to a composition for smoothing the appearance of wrinkles in the skin, a composition for promoting lipid formation and a composition for improving skin tenacity, wherein the composition comprises the aforementioned compound, and to a method for reducing wrinkles or related skin signs of aging and a method for improving skin tenacity with the use of the composition.

The present invention also relates to a novel bleaching composition. More specifically, the invention relates to a bleaching composition with melatonin as an active ingredient. In addition, the invention can contain vitamin C or vitamin E as an active ingredient. The invention further relates to a composition for diminishing the appearance of blemishes in the skin, wherein the composition comprises the aforementioned compound. The invention also relates to a method for diminishing blemishes or related skin signs of aging through use of the composition.

### BACKGROUND

### Increasing Cellular Fat

Keratinocytes are stratified, squamous epithelial cells found in the skin and mucosa, including oral, esophageal, corneal, conjunctival, and genital epithelia. Keratinocytes provide a protective barrier between host and environment, functioning to prevent substances from the environment from entering the host as well as prevent the loss of important host components to the environment. Keratinocytes undergo differentiation as they progress from the basal layer of the basement membrane to the surface of the skin.

The peroxisome proliferator-activated receptors (PPARs) are a family of nuclear hormone receptors that function as ligand-gated transcription factors to facilitate activation and repression. PPARs are involved in a variety of physiological processes and various aspects of metabolic disease. PPARs have been shown to be transcriptionally activated by a diverse group of compounds, including clifibrate-related hypolipidemic drugs, the synthetic arachidonate analog ETYA (5,8,11,14-eicosatetraynoic acid) the leukotriene antagonist LY-171883 and polyunsaturated fatty acids. See Tonotonoz et al. “Stimulation of Adipogenesis in Fibroblasts by PPARγ2, a Lipid-Activated Transcription Factor” 1994, Cell 79:1147-1156. The first member identified in the PPAR family (PPARα) responded to a compound that induced peroxisome proliferation. The subsequent family members, such as PPARβ, PPARγ, PPARδ, although not involved in a similar peroxisome proliferation process, act as regulators of lipid metabolism and metabolic control. Specifically, peroxisome proliferator-activated receptor gamma (referred to herein as “PPARγ”) has been shown to modify keratinocyte and adipose differentiation, modulate metabolism and inflammation in immune cells as well as possessing strong antigrowth properties and inhibition of angiogenesis. The activation of PPARγ is primarily through ligand binding (endogenous ligands such as fatty acids or eicosanoids or exogenous ligands such as discussed below) which in turn activates the receptor. The identification of exogenous and endogenous PPARγ ligands, has resulted in exploration into the potential uses for this class of drugs in the treatment of cardiovascular disorders and cancer.

A group of antidiabetic and anti-inflammatory agents called thiazolidinediones (e.g. pioglitazone, troglitazone and rosiglitazone) are known PPARγ agonist ligands that function to increase insulin sensitivity and are primarily used in the treatment of type 2 diabetes. PPAR modulate glucose metabolism and insulin sensitivity, thereby reducing plasma glucose and insulin levels in type 2 diabetes. Specifically, one of the thiazolidinediones, troglitazone, acts as an agonist ligand to both PPARα and PPARγ, with higher affinity for PPARγ.

Skin Bleaching

The aging process has multiple effects on the overall thickness and elasticity of the skin. As skin ages, the amount of collagen produced is decreased and the type of collagen changes. In addition, the elastin in the skin decreases, the melanin granules collect into areas of dark-colored blemishes, the cells of the skin become older and the layers of expired cells increases. There are some remedies available to address these problems. Traditionally, retin-A is used to increase collagen production, decrease elastin loss, decrease production of metalloproteases (which may cause oxidative damage to the skin), disperse melanin granules and exfoliate the layers of dead skin cells from the skin. However, retin-A has some undesirable side effects and requires monitoring of sun exposure while treating skin. There are additional treatments for aging skin such as hydroquinone (bleaches skin and slows melanin production), alpha hydroxy acid (acts similar to retin-A), anti-oxidants (e.g. Cellex-C, Prevage or Revale). There is a continuing need for the development of skin care products that aid in the appearance of younger, more vibrant, healthy looking skin.

Melatonin is a naturally occurring hormone that has varied expression during the daily cycle. Most commonly, it is know that melatonin affects the circadian rhythms of biological functions, in addition to its role as an antioxidant and its protection of DNA.

### SUMMARY OF THE INVENTION

Compositions are disclosed for cosmeceuticals that promote an increase in lipid production in keratinocytes. Methods for preparing comeceutical compositions resulting in the promotion of an increase in lipid production in keratinocytes are also disclosed.

Compositions are also disclosed for cosmeceuticals that diminish the appearance of skin blemishes. Methods for preparing cosmeceutical compositions resulting in a reduction of skin blemishes are also disclosed.

In one aspect of the invention, the composition comprises at least one peroxisome proliferator-activated receptor gamma agonist in a cosmeceutically acceptable medicum.

In another aspect, the composition comprises troglitazone.

In yet another aspect, the composition comprises troglitazone and fibroblast growth factors.

In an additional embodiment, the composition is topically administered or can also be formulated as a leave-on product.

In an additional aspect, the composition contains about 0.0005% to 0.5% by weight peroxisome proliferator-activated receptor gamma agonist.

In another aspect, the composition contains about 0.0005% to 0.5% by weight peroxisome proliferator-activated receptor gamma agonist.

In yet another aspect, the composition contains about 0.005% to 0.05% by weight peroxisome proliferator-activated receptor gamma agonist.

In another embodiment, the composition comprises at least one peroxisome proliferator-activated receptor gamma agonist and functions to promote lipid formation.

Another aspect is a method of increasing lipid production in keratinocytes comprising the step of: topically administering a composition comprising a cosmeceutically effective amount of a peroxisome proliferator-activated receptor gamma agonist to a person in need thereof.

An additional aspect is a method of increasing lipid production in the skin comprising activation of peroxisome proliferator-activated receptor gamma with a composition containing a) a cosmeceutically acceptable medium and b) a cosmeceutically effective amount of at least one peroxisome proliferator-activated receptor gamma agonist, wherein said agonist has an affinity for peroxisome proliferator-activated receptor gamma.

Another aspect of the invention is a method of increasing lipid production in the skin with a composition comprising troglitazone.

Another embodiment is a composition comprising: a) a cosmeceutically acceptable medium and b) a cosmeceutically effective amount of melatonin, wherein the composition promotes lightening of the skin.

An additional embodiment is a composition comprising about 0.3% by weight melatonin.

An additional embodiment is a method of diminishing the appearance of darkened skin blemishes comprising the step of topically administering a composition comprising melatonin.

An additional embodiment is a composition comprising: a) a cosmeceutically acceptable medium, b) a cosmeceutically effective amount of at least one peroxisome proliferator-activated receptor gamma agonist and c) a cosmeceutically effective amount of melatonin.

Another aspect of the invention is a composition comprising about 0.005% by weight of peroxisome proliferator-activated receptor gamma agonist.

An additional aspect is a composition comprising about 0.15% by weight of melatonin.

An additional aspect is a composition comprising peroxisome proliferator-activated receptor gamma agonist and melatonin that is topically administered or formulated as a leave-on product.

Another aspect of the invention is a composition comprising peroxisome proliferator-activated receptor gamma agonist, melatonin and fibroblast growth factor.

### DESCRIPTION OF THE DRAWINGS

These and other advantages of the present invention will be readily understood with reference to the following specifications and attached drawings wherein:

FIG. 1 is a photograph of keratinocytes treated with troglitazone and stained for lipids.

FIG. 2 is a photograph of human preadipocytes treated with troglitazone and stained for lipids.

FIG. 3 is a photograph of human preadipocytes treated with troglitazone and stained for lipids.

### DETAILED DESCRIPTION

Preferred embodiments of the present invention will be described hereinbelow with reference to the accompanying drawings. In the following description, well-known functions or constructions are not described in detail because they would obscure the invention in unnecessary detail.

PPARγAgonists and Lipid Production

In one aspect, the present invention is based on the discovery that drugs, such as PPARγ agonists, like troglitazone, affect the expression of genes in the lipid metabolic pathways and in turn cause an increase in the production of lipids in keratinocytes. Surprisingly, the topical administration of troglitazone has been shown to increase the lipid droplets in keratinocytes when compared to untreated cells. The response of keratinocytes to treatment with troglitazone over a 48 hour treatment period results in an increase in lipid production of keratinocytes.

PPARs have been shown to induce enzymes of the peroxisomal fatty acid β-oxidation system and activate transcription of the acyl-CoA oxidase gene (which catalyzes the rate-limiting step in the β-oxidation pathway). Activation of PPARγ (also known as NR1C3 based on its relationship to the steroid/thyroid hormone superfamily of nuclear receptors) leads to increased fat storage and secretion of insulin-sensitizing adipocytokines such as adiponectin from adipocytes. PPARγ is the functional receptor for thiazolidinediones (TZDs), a class of insulin-sensitizing drugs which are used in the treatment of type 2 diabetes mellitus.

PPARγ has also been shown to be involved in differentiation of other cells and tissues, such as macrophages, breast, and colon, and mutations of PPARγ that destroy receptor function have been found in sporadic human colon cancer. PPARγ functions as a heterodimer with retinoid X receptor (RXR) and binds (as a PPARγ/RXR complex) to sequences (DR-1 sites) and activates transcription upon ligand binding. PPARγ binds and is activated by the synthetic TZD ligands, and natural ligands such as fatty acid derivatives (15-deoxy-prostaglandin linoileic acid, PUFAs eicosapentaenoic acid (EPA) and docosahexaenoic acid (DHA), and 9(S)-hydroxy-octadecadienoic acid (HODE), and several hydroxyeicosatetraenoic acids (HETEs)). See Rosen et al. “PPARγ Is Required for the Differentiation of Adipose Tissue In Vivo and In Vitro” 1999, Molecular Cell, 4:611-617. PPARγ ligands have also been shown to upregulate fibroblast growth factor 2 (FGF2). See Yasuda et al. “PPAR-γligands up-regulate basic fibroblast growth factor-induced VEGF release through amplifying SAPK/JNK activation in osteoblasts” 2005. Biochemical and Biophysical Research Communications” 328 (1):137-143

PPARγ also plays a role in adipogenesis in early stages, following increases in the transcription factors, CCAAT/enhancer-binding protein (“C/EBP”), C/EBPβ and C/EBPδ. PPARγ has two isoforms, PPARγ1 and PPARγ2, which are both thought to be competent to stimulate adipogenesis. PPARγ expression results in increases in C/EBPα, which promotes the differentiated phenotype, at least in part by increasing the expression of PPARγ via positive feedback. In a variety of gain-of-function experiments, PPARγ has been shown to be sufficient to induce adipogenesis in a variety of cell types. In addition, chimeric mouse analysis has shown PPARγ is essential to the process of adipogenesis and may play a role in differentiation of other tissues as well (e.g. sebocytes, macrophages, and colon and breast epithelium). See Rosen et al. 1999. Without being limited to any particular theory, it is thought that PPARγ and C/EBPα could interact to stimulate adipose tissue by activation of the complete adipocyte gene expression, although this may occur in a temporally regulated manner, with PPARγ functioning to commit primitive mesodermal cells to the adipose lineage and activate expression of early genes and C/EBPα functioning later in the differentiation process for expression of late genes responsible for triggering arrest, terminal differentiation, lipid accumulation, or some combination of these. See Tontonoz et al. 1994; Tontonoz et al. “Fat and Beyond: The Diverse Biology of PPARγ” 2008, Annu. Rev. Biochem. 77:289-312.

PPARγ operates at the center of the adipogenic cascade in a feedback loop with C/EBPα. It participates in a variety of signaling pathways operating CCAAT/enhancer-binding protein upstream of PPARγ, including the hedgehog and Wnt pathways, C/EBPβ and -δ, and EBFs. During adipocyte differentiation several upstream genes are thought to be required for the activation of the PPARG gene. These include the CCAAT/enhancer-binding proteins β and δ (C/EBPβ and C/EBPδ), SREBP-1c, Krüppel-like factor-5 (KLF5), KLF15, zinc-finger protein 423 (Zfp423), and early B-cell factor (Ebf1). In the process of adipocyte differentiation PPARγ activates nearly all of the genes required for this process. These genes include aP2 which is required for transport of free fatty acids (FFAs) and perilipin which is a protein covering the surface of mature lipid droplets in adipocytes. Additional genes regulated by PPARγ that are involved in lipid metabolism or glucose homeostasis include lipoprotein lipase (LPL), acyl-CoA synthase (ACS), acetyl-CoA acetyltransferase (ACAT), several phospholipase A (PLA) genes, the adipocytokine adiponectin, the gluconeogenic enzyme PEPCK, and glycerol-3-phosphate dehydrogenase (GPDH). PPARγ also functions in macrophage lipid metabolism by inducing the expression of the macrophage scavenger receptor, CD36.

Troglitazone is a member of the thiazolidinedione class of drugs, an anti-diabetic and antiinflammatory drug. Other members of this drug family include: pioglitazone and rosiglitazone; which, like troglitazone, have all been shown to activate PPARs. Troglitazone is a ligand to both PPARγ and PPARγ (binding more strongly to PPARγ). Structurally, troglitazone [(RS)-5-(4-[(6-hydroxy-2,5,7,8-tetramethylchroman-2-yl)methoxy]benzyl)thiazolidine-2,4-dione; molecular weight 441.541 g/mol] contains an α-tocopheroyl moiety, potentially giving it vitamin E-like activity in addition to its PPAR activation:

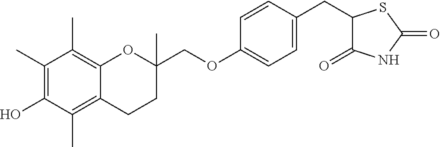

It has been shown to reduce inflammation: troglitazone use has been associated with a decrease of nuclear factor kappa-B (NF-κB—an important cellular transcription regulator for the immune response) and a concomitant increase in its inhibitor (IκB). The mechanism of action of the TZDs is a function of the activation of PPARγ and the consequent induction of genes necessary for differentiation of adipocytes.

The observed increase in lipid production upon treatment with troglitazone has many desired effects in management of skin and skin disorders, including anti-ageing, anti-wrinkle and/or an anti-cellulite effects, minimizing the appearance of wrinkles, blemishes, skin lines, oily skin, acne, dry skin, xerosis, ichthyosis, dandruff, brownish spots, keratosis, melasma, lentigines, age spots, dark circles around eyes, skin pigmentation, topical inflammation, liver spots, pigmented spots, wrinkles, blemishes, skin lines, oily skin, acne, warts, eczema, pruritic skin, psoriasis, inflammatory dermatoses, disturbed keratinization, dandruff, bacterial infection, fungal infection, wound healing, body odor, and skin changes associated with aging.

Additionally, the present invention is based on the discovery that hormones, such as melatonin, affect the coloration of darkened spots on the surface of the skin and can diminish the appearance of such spots when placed in a cosmeceutical composition. In another aspect of the present invention, the addition of vitamin C or vitamin E to the composition also promotes the reduction of darkened skin spots and promotes bleaching of the skin.

The observed decrease in the appearance of darkened skin blemishes upon treatment with a melatonin composition has many desired effects in management of skin and skin disorders, including anti-ageing, minimizing the appearance of wrinkles, blemishes, dry skin, xerosis, ichthyosis, dandruff, brownish spots, keratoses, melasma, lentigines, age spots, dark circles around eyes, skin pigmentation, topical inflammation, liver spots, pigmented spots, skin lines, oily skin, acne, warts, eczema, pruritic skin, psoriasis, inflammatory dermatoses, disturbed keratinization, dandruff, bacterial infection, fungal infection, wound healing, body odor, and skin changes associated with aging.

Accordingly, a preferred composition of the invention contains a PPARγ agonist in the presence of melatonin in a cosmeceutically acceptable medium/vehicle.

Cosmetically Acceptable Composition

The composition according to the invention also comprises a dermatologically/cosmetically acceptable vehicle to act as a diluent, dispersant or carrier for the PPARγ agonist and/or melatonin. The vehicle can comprise materials commonly employed in skin care products such as water, liquid or solid emollients, silicone oils, emulsifiers, solvents, humectants, thickeners, powders, propellants and the like. Examples of vehicle ingredients include water, glycerin, hydrogenated polyisobutene, cetearyl alcohol, ceteareth-20, macadamia integrifolia seed oil (macademia nut oil), dimethicone, tocopheryl acetate, stearoxytrimethylsilane, stearyl alcohol, panthenol, farnesol, benzyl alcohol, phenoxyethanol, acrylates/C10-30 alkyl acrylate crosspolymer, sodium hydroxide, citric acid for lotions or water, petrolatum, glyceryl polymethacrylate, dicaprylyl ether, glycerin, dimethicone, glyceryl stearate, cetyl alcohol, prunus amygdalus dulcis (sweet almond) oil, PEG-30 glyceryl stearate, tocopheryl acetate, benzyl alcohol, phenoxyethanol, sodium hydroxide, acrylates/C10-30 alkyl acrylate crosspolymer, disodium EDTA, propylene glycol for creams. A preferred vehicle is Cetaphil®. Other agents which can be employed in the present application in the dermatologically acceptable vehicle include fibroblast growth factor (e.g. basic fibroblast growth factor, fibroblast growth factor 2); acetyl hexapeptide, tromethamine, glutathione peroxidase, grapefruit, glycolic acid, heparin sulfate, acetyl hexapeptide, tromethamine, glycolic acid, sphingoid and phospholipid derivatives (e.g. ceramides, phytosphingosine, sphingosine, pseudoceramides, phospholipids, lysophospholipids); antioxidants (e.g. glutathione) and vitamins [e.g. tocopherol and derivatives, ascorbic acid and derivatives, niacinamide and derivatives, vitamin complexes, alpha-lipoic acid, retinol and derivatives, panthenol, vitamin C (including vitamin C derivatives), vitamin E; antiinflammatories (e.g. bisabolol, allantoin, phytantriol), Coenzyme Q10, Idebenone; botanical agents such as polyphenolics, flavonoids or isoflavones]; moisturizing agents (e.g. amino acids, hyaluronic acid and derivatives, creatine and derivatives, trimethylglycine, myoinositol, pyroglutamatic acid and derivatives, taurine, guanidine and derivatives and hydroxy acids); skin whitening agents (e.g. kojic acid, arbutin, hydroquinone); peptides, modified peptides, protein hydrolysates, caffeine and sunscreens and UV absorbers.

Formulation Tables

TABLE I
Troglitazone (FW = 441.55)
5 mg in 50 ul (0.1 mg/ul) Stock Solution

| | | Total | | Amount | |
|---|---|---|---|---|---|
| Desired | Desired | Volume/ | Amount | Active | |
| Conc | Percent | Mass | Active | Ingredient | |
| (M) | Conc | Desired | Ingredient | (mgs) | Amount of Stock |
| 0.0001 | 0.01 | 20 gms | 0.002 gms | 2 mgs | 20 uls of the 0.1 |
| 0.00005 | 0.005 | 20 gms | 0.001 gms | 1 mgs | 10 uls of the 0.1 |

TABLE II
Melatonin (FW = 232.28)
1 g in 5 ml (0.2 mg/ul)

| | | Total | | Amount | |
|---|---|---|---|---|---|
| | Desired | Volume/ | Amount | Active | |
| Desired | Percent | Mass | Active | Ingredient | |
| Conc. | Conc. | Desired | Ingredient | (mgs) | Amount of Stock |
| 0.005 | 0.5 | 20 gms | 0.1 gms | 100 mgs | 500 uls of the 0.2 |
| 0.001 | 0.1 | 20 gms | 0.02 gms | 20 mgs | 100 uls of the 0.2 |

The cosmeceutically effective amount of a PPAR agonist that is used in the cosmeceutically acceptable composition has a concentration of about 0.5% to 0.0001% PPAR agonist preferably from about 0.1% to 0.0005%, and most preferably about 0.05% to 0.005%. The cosmeceutically effective amount of a PPAR agonist is partially dependent on the cells or the individual being treated and higher concentrations may be necessary to achieve desired results, in addition higher concentrations may result in increased side effects. The Formulation Table shows exemplary calculations for producing products of 0.01% and 0.005% PPAR agonist, similar calculations can be used to produce a cosmeceutically acceptable composition at the desired concentration.

The cosmeceutically effective amount of melatonin that is used in the cosmeceutically acceptable composition has a concentration of about 0.5% to 0.00001% melatonin. In one embodiment, the preferred melatonin concentration is about 0.3%. In another embodiment, the preferred melatonin concentration is about 0.0015%. The cosmeceutically effective amount of melatonin is partially dependent on the cells or the individual being treated and higher concentrations may be necessary to achieve desired results, in addition higher concentrations may result in increased side effects. The Formulation Table shows exemplary calculations for producing products of 0.1% and 0.5% melatonin, similar calculations can be used to produce a cosmeceutically acceptable composition at the desired concentration

The dermatologically acceptable vehicle will usually form from about 80% to about 99.999%, preferably from about 90% to about 99.995% and most preferably about 99.99% by weight of the composition, and can, in the absence of other cosmetic adjuncts, form the balance of the composition. In a preferred embodiment, the troglitazone is maintained at a concentration of about 0.005% or 0.01% by weight in a cosmeceutically acceptable medium (e.g. about 10 gms of vehicle, with about 0.5 mg or 1 mg troglitazone, respectively). In another preferred embodiment, the melatonin is maintained at a concentration of about 0.03% by weight in a cosmeceutically acceptable medium. In yet another embodiment, melatonin is maintained at a concentration of about 0.15% by weight in a cosmeceutically acceptable medium in combination with troglitazone at a concentration of about 0.005% by weight. In another preferred embodiment, vitamin C or vitamin E are also included in the compositions and are maintained at a therapeutically effective vitamin concentration of about 0.5% in a cosmeceutically acceptable medium.

The skin care formulations can be an aqueous solution, a water-in-oil (w/o) emulsion, an oil-in-water (o/w) emulsion, a dispersion of lipids, an aqueous, water-alcohol, oil or oil-alcohol gel, a solid stick, a wet-wipe or an aerosol. If the dermatologically acceptable vehicle itself is an (w/o) or (o/w) emulsion, it can contain 5 to 50% of an oilphase and 47 to 94.95% water, with respect to the weight of the whole formulation.

Product Preparation, Form, Use and Packaging

To prepare the topical composition according to the present invention, the usual manner for preparing skin care products may be employed. The active components are generally incorporated in a dermatologically acceptable carrier in conventional manner. The active components can suitably be dissolved or dispersed in a portion of the water or another solvent or liquid to be incorporated in the composition. The preferred compositions are oil-in-water or water-in-oil emulsions.

The composition may be in the form of conventional skin-care products such as a cream, gel or lotion or the like. The composition can also be in the form of a so-called “rinse-off” product, e.g., a bath or shower gel, possibly containing a delivery system for the actives to promote adherence to the skin during rinsing. Most preferably, the product is a “leave-on” product; a product to be applied to the skin without a deliberate rinsing step soon after its application to the skin. The composition can be formulated for night creams with a thicker delivery system or with variations in the amount of the PPAR agonist or melatonin for day creams formulated with less PPAR agonist or melatonin in a thinner delivery system.

The inventive composition may be packaged in any suitable manner such as in a jar, a bottle, tube, roll-ball, or the like, in the conventional manner.

The active ingredients described in the present invention may be applied one or more times daily to the portion of skin requiring treatment. The improvement in skin appearance will usually become significantly visible by about two weeks, depending on the status of the initial skin condition, the concentration of the active components used in the composition, the volume of composition used and the frequency of application.

In one embodiment, a small quantity, about 0.25 ml, of the composition is applied to the skin from a suitable container or applicator and spread over and/or rubbed into the skin using the hands or fingers or a suitable device. The composition is formulated as a “leave-on” product and does not require any gloves or special applicators for effective use. Once applied to the skin in the affected area, the composition will begin to elicit the desired effect and promote lipid production.

### Example 1

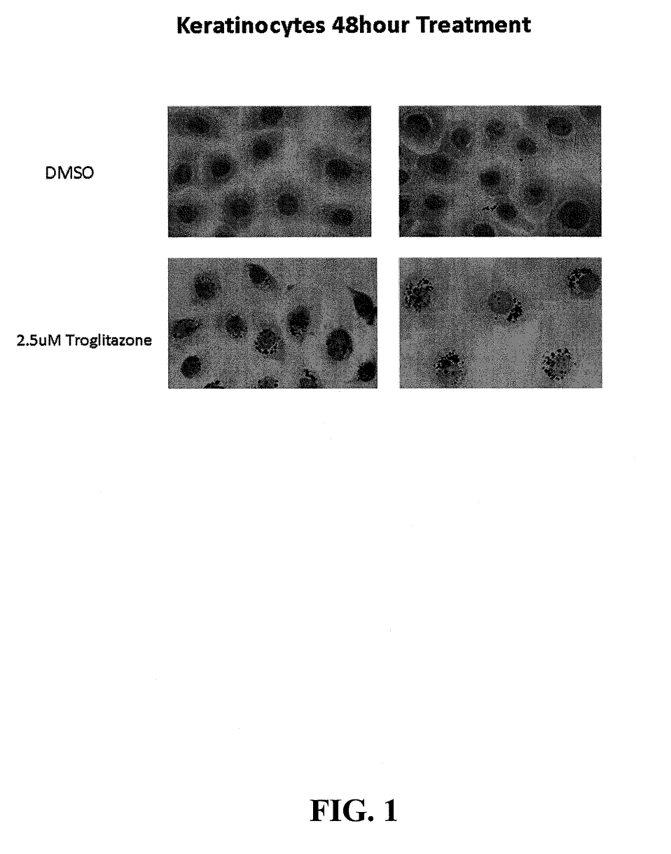

As shown in FIG. 1, keratinocytes treated with of 2.5 uM troglitazone for 48 hours show an increase in lipid production in the cells, evidenced by the lipid droplets stained with Oil Red O as compared to the absence and/or low prevalence of lipid droplets in the DMSO control.

### Example 2

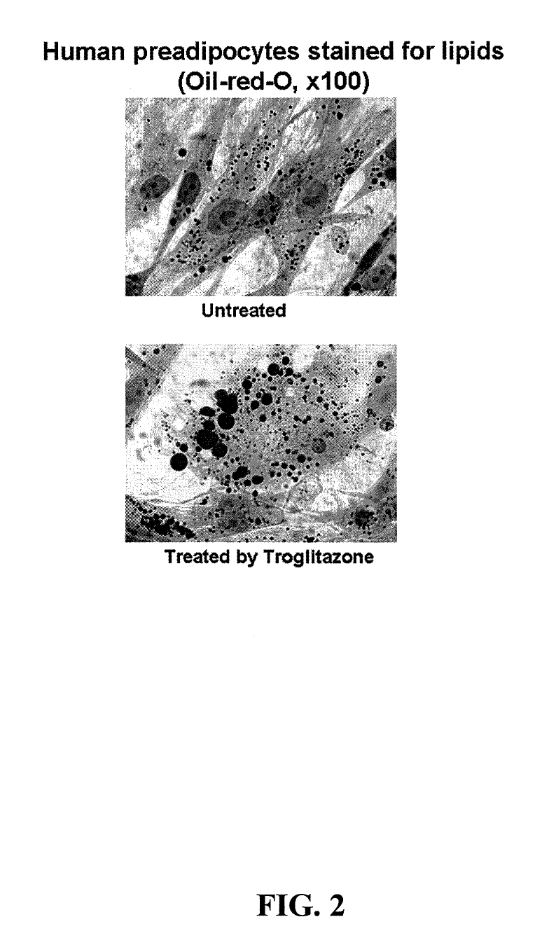

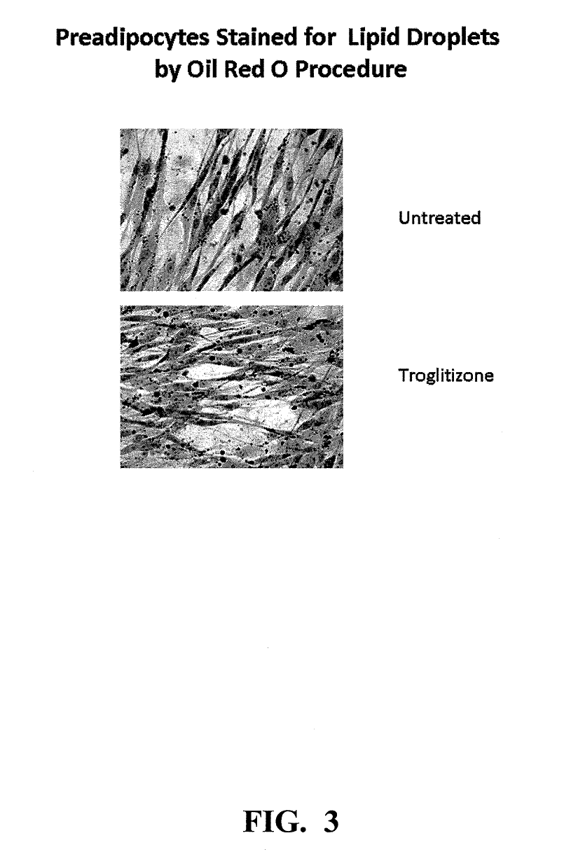

As shown in FIG. 2, preadipocytes treated with of 1.1 uM troglitazone for about 48 hours show an increase in the lipid production (quantity and the size of lipid droplets) as evidenced by the lipid droplets stained with Oil Red O. Increases in lipid droplet after troglitazone treatment is also seen in FIG. 3.

### Example 3

### Anti Wrinkle Day Cream

Cream for beautiful youthful skin while at the same time reducing tissue damage. Significant results in only two weeks. Use daily, directly into clean skin. The cream may oxidize and darken with time but will remain effective throughout use.

TABLE III

| Anti Wrinkle Day Cream - New | | |
|---|---|---|
| Generation | Amount | |
| Troglitazone | 0.5 | milligram |
| Vitamin C | 500 | milligram |
| Fibroblast Growth Factor 2 | 0.5 | micrograms |
| Tromethamine | 0.5% | |
| Glycolic Acid | 2% | |
| Vehicle | 10 | grams |

### Example 4

### Anti Spot Cream

Anti Spot formulation can diminish up to 90% of skin hyper pigmentation. The integration of natural components such as pro antioxidants and activator factors found in human cells into the formulation, allows cream to clarify birth marks, sun spots, age marks, hormonal induced hyperpigmentation and much more. Unlike other skin lighteners formulated with bleaching ingredients or hydroquinone, Perfektion's Anti Spot does not increase the risk of skin damage from sun exposure. Use twice a day applied directly to designated areas. The cream may oxidize and darken with time but will remain effective throughout use.

TABLE IV

| Antispot | Amount per 10 gms | |
|---|---|---|
| Melatonin | 30 | milligrams |
| Vitamin C | 500 | milligrams |
| Tromethamine | 0.5% | |
| VITtamin E | 500 | milligrams |
| Grapefruit | 0.50% | |
| Glutathione Paroxide | 30 | milligrams |
| Vehicle | 10 | grams |

### Example 5

### Reconstruction Night Cream

Reconstruction Night Cream regenerates tissue damage and substantially diminishes signs of age in only two weeks. Composition stimulates cells to enhance production of fundamental ingredients found in human cells that rejuvenate and maintain the healthy natural appearance of your skin. Reconstruction-Night Cream also stops the appearance of facial wrinkles. Apply every evening directly over clean skin. The cream may oxidize and darken with time but will remain effective throughout use.

TABLE V

| Reconstruction-Night- | | |
|---|---|---|
| Cream | Amount per 10 gms | |
| Troglitazone | 0.5 | milligrams |
| Vitamin C | 500 | milligrams |
| Fibroblast Growth Factor 2 | 1.5 | micrograms |
| Acetyl Hexapeptide | 40 | milligrams |
| Tromethamine | 0.50% | |
| Glycolic Acid | 2% | |
| Vehicle | 10 | grams |

### Example 6

### Reconstruction Liquid Serum

TABLE VI

| Reconstruction Liquid Serum | Amount per 10 gms | |
|---|---|---|
| Troglitazone | 1 | milligrams |
| Vitamin C | 500 | milligrams |
| Fibroblast Growth Factor 2 | 1.5 | micrograms |
| Acetyl Hexapeptide | 40 | milligrams |
| Tromethamine | 0.50% | |
| Glycolic Acid | 2% | |
| Acetyl Hexapeptide | 80 | milligrams |
| Distillated Water | 10% | |
| Vehicle | 10 | grams |

### Example 7

### Troglitazone/Melatonin Cream (Eye Cream)

TABLE VII

| Eye Cream | Amount per 10 gms | |
|---|---|---|
| Troglitazone | 0.5 | milligrams |
| Vitamin C | 500 | milligrams |
| Fibroblast Growth Factor 2 | 1.5 | micrograms |
| Acetyl Hexapeptide | 40 | milligrams |
| Tromethamine | 0.50% | |
| Glycolic Acid | 2% | |
| Melatonin | 15 | milligrams |
| Vehicle | 10 | grams |

### Additional Formulation Examples

A 0.1 mg/ul solution of Troglitazone is made in DMSO. To make a 0.005% by weight composition, an aliquot of 100 uls of the 0.1 mg/ul Troglitazone solution is added to 200 grams of lotion or cosmeceutically acceptable medium and mixed thoroughly. To make a 0.0005% by weight composition, an aliquot of 10 uls of a 0.1 mg/ul Troglitazone solution is added to 200 grams of lotion or cosmeceutically acceptable medium and mixed thoroughly. Additional concentrations can be made from a concentrated stock solution by methods known to one of ordinary skill in the art. The cosmeceutical formulation (lotion) can be stored at ambient temperature for topical use on those areas of the skin wherein additional lipid production is desired.

A 0.1 mg/ul solution of Melatonin is made in DMSO. To make a 0.1% by weight composition, an aliquot of 100 uls of the stock solution is added to 200 grams of lotion or cosmeceutically acceptable medium and mixed thoroughly. Additional concentrations can be made from a concentrated stock solution by methods known to one of ordinary skill in the art. The cosmeceutical formulation (lotion) can be stored at ambient temperature for topical use on those areas of the skin wherein additional lipid production is desired.

While the present invention has been described with respect to what is presently considered to be the preferred embodiments, it is to be understood that the invention is not limited to the disclosed embodiments. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

All U.S. and foreign patent documents, all articles, brochures, and all other published documents discussed above are hereby incorporated by reference into the Detailed Description of the Preferred Embodiment.

What is claimed is:

1. A composition comprising: a) a cosmeceutically acceptable medium and b) a cosmeceutically effective amount of at least one peroxisome proliferator-activated receptor gamma agonist.

2. A composition according to claim 1, wherein said peroxisome proliferator-activated receptor gamma agonist is troglitazone.

3. A composition according to claim 1, further comprising fibroblast growth factor.

4. A composition according to claim 1, wherein said composition is topically administered.

5. A composition according to claim 1, wherein said composition is formulated as a leave-on product.

6. A composition according to claim 1, wherein the cosmeceutically effective amount consists of about 0.0005 to 0.5 weight percent of the composition.

7. A composition according to claim 1, wherein the cosmeceutically effective amount consists of about 0.005 to 0.01 weight percent of the composition.

8. A composition comprising: a) a cosmeceutically acceptable medium and b) a cosmeceutically effective amount of at least one peroxisome proliferator-activated receptor gamma agonist, wherein said agonist promotes lipid formation.

9. A method of increasing lipid production in keratinocytes comprising the step of: topically administering a composition comprising a cosmeceutically effective amount of a peroxisome proliferator-activated receptor gamma agonist to a person in need thereof.

10. A method of increasing lipid production in the skin comprising activation of peroxisome proliferator-activated receptor gamma with a composition containing a) a cosmeceutically acceptable medium and b) a cosmeceutically effective amount of at least one peroxisome proliferator-activated receptor gamma agonist, wherein said agonist has an affinity for peroxisome proliferator-activated receptor gamma.

11. A method according to claim 13, wherein said agonist is troglitazone.

12. A composition comprising: a) a cosmeceutically acceptable medium and b) a cosmeceutically effective amount of melatonin, wherein the composition promotes lightening of the skin.

13. A composition according to claim 16, wherein the cosmeceutically effective amount is about 0.3% weight percent of the composition.

14. A method of diminishing the appearance of darkened skin blemishes comprising the step of topically administering a composition of claim 16 to a person in need thereof.

15. A composition comprising: a) a cosmeceutically acceptable medium, b) a cosmeceutically effective amount of at least one peroxisome proliferator-activated receptor gamma agonist and c) a cosmeceutically effective amount of melatonin.

16. A composition according to claim 15, wherein the cosmeceutically effective amount of peroxisome proliferator-activated receptor gamma agonist is about 0.005 weight percent of the composition.

17. A composition according to claim 15, wherein the cosmeceutically effective amount of melatonin is about 0.15% weight percent of the composition.

18. A composition according to claim 15, wherein said composition is topically administered.

19. A composition according to claim 15, wherein said composition is formulated as a leave-on product.

20. A composition according to claim 15, further comprising fibroblast growth factor.
